(19) United States
(12) Patent Application Publication
(10) Pub. No.: US 2013/0130329 A1
(43) Pub. Date: May 23, 2013

(54) METHOD FOR EXTRACTING SUBSTANCES FROM SOAPBERRY FRUIT AND ITS SEEDS

(71) Applicant: Heng-Jui Hsu, Taipei (TW)

(72) Inventor: Heng-Jui Hsu, Taipei (TW)

(21) Appl. No.: 13/742,288

(22) Filed: Jan. 15, 2013

Related U.S. Application Data
(63) Continuation-in-part of application No. 12/457,398, filed on Jun. 10, 2009.

(51) Int. Cl. C12P 19/02; C12P 19/04

(52) U.S. Cl. CPC C12P 19/02; C12P 19/04
USPC 435/101; 435/105

(57) ABSTRACT

An exclusive method for extracting active interface saponin and organic substances from soapberry; organic elements and oleic alcohol products from soapberry seeds through fermenting process, and end products made therefrom. By this method, every part of the soapberry is processed to become the raw material of varies of products and daily necessaries. Said method is toxin free and biologically safe, produce no solid and liquid wastage, zero carbon emissions, zero chemical pollution, low energy consumption and ecologically friendly. The end products produced by present invention are variables which can be applied in cosmetics, medicals, cleaning products, skin caring products and so on, thus, are with excellent economic value and industrial viability in mass production.

Present invention is a CIP (Continuation-In-Part) application of application Ser. No. 12/457,398 filed Jun. 9, 2009.

### FIELD OF THE INVENTION

### Background of the Invention

Soapberry (Sapindus mukorossii Gaertn) plant is a woody plant with a tall trunk. The fruit thereof is known as soapberry nut. Soapberry is a cash crop. Its scientific value was discovered by western pioneers who traveled to India and South East Asia. Botanists at that time called Soapberry as “the soap of the Indians”.

The usage of soapberry was recorded and described in The Romance of Formosa published in 1685. The soapberry tree was referred as “Yellowed-eyes Tree” whose fruit was described as “a yellow wrinkled fruit used for washing clothes which functions like soap.” Bubbles are formed when rubbing the skin of the fruit in water. Ancient Asian has been using it to wash clothing for several centuries. However, due to the yellow pigments contained within the skin, clothing will turn yellow after several rounds of washing. Soapberry was also believed to keep ones hair black, silky, clean and soft. In addition to saponins, soapberry contains soluble fats; pots and utensils washed with soapberry will become shiny. By this reason, the present jewelry industry still uses it to clean jewelry.

For centuries, fruits of soapberry trees were picked, crushed, molded into lumps and made into soap until the petrochemical synthetic detergent was manufactured. Due to the low cost and high yield of cleansers made of petrochemical interface active agents, the use of traditional sapindus soap in our daily life has gradually disappeared. The use of soapberry has dwindled and as a result, most soapberry trees were cut down. However, people are now discovering the seriousness of the pollution caused by inorganic chemicals, which have contaminated our environment. A new environmental awareness has given rise to environmentally friendly biotechnology, while the increasing cost of petrochemicals marks a starting point for a new era of bio-technology, in which soapberry is regaining its past popularity.

Sapindus is enriched with botanical saponin which can be used as natural soap or medicinal plant. The flesh of soapberry is rich in Sapindoside A, B, C, D, and E. Its elementary anhydride is known as hederagenin, which also contain rutin and plentiful vitamin C, tyrosine, glycine, alanine, fructose, glucose, sugar E, E-sugar, Arab sugar, rhamnose, and so on.

Saponin is the most important ingredient in soapberry flesh, although many plant species contain an abundant amount of saponin, they all differ in some way. Ginseng, for example, produces ginsenoside, and tea produces tea saponin, both of which have therapeutic properties. The saponin content of soapberry pulp stands at 37%—the highest percentage so far discovered in a plant.

Saponin exists in a wide variety of plants. At present, over 50% of plant life is known to contain saponin; in animals, the starfish is the only creature known to contain saponin.

Biosynthesis saponin in soapberry primarily acts to help the organism defense itself against viruses, bacteria, fungi, insects, mollusks, and used to promote healing after such attacks.

Saponin is known as the pre-elements of hormone in chemical structure, and is believed to play the role of nerve conductor, and has the ability to help the organism adjust to the environment.

The flesh of soapberry fruit is rich in natural botanical saponin, and can therefore be used directly as soap. Soapberry is also a very important medicinal plant that produces an active interface agent which can serve as an industrial emulsifiers, moistening agent, or foaming agent.

The fruit of soapberry also contains high sugar content, organic amino acid, active interface, and is rich in bubble-making properties—soft and thick bubbles are formed once the pulp of the soapberry is placed and rubbed in the water. Therefore, as recorded in the Compendium of Materia Medica, since the time of immemorial soapberry has been used as cleanser of various sorts. It has been used as hair tonic, in oral hygiene and maintenance, general health care and so on.

The extracting process for Soapberry saponin is simple: first collect the soapberry nuts, dry or crush them, and then stew them in boiling water till the saponin dissolve into the water. However, the overall productivity of such a method is very low, and more importantly, such a method is unable to extract the anhydrite completely.

As a result, some manufacturers have to continuously improved methods of saponin extraction. Such as disclosed in the cited reference No. 1, with publication No. TW 200641122 entitled “A Method to Purify Saponin Using Ion-exchange and Reverse Osmosis”, which the process is translated into English: first, strong ion-exchange resins are used as raw materials. By carrying out an ions-exchange process, the organic acid of soapberry is thus removed. This is then followed by a reverse osmosis to remove excessive water content to obtain pure saponin extract. After the ion-exchange process, an optional weak ion-exchange process can then be carry out to remove its sugar content, follow by another reverse osmosis.

Another method to extract saponin as disclosed in cited reference No. 2 with publication No. TW 200800246 entitled “Method for Producing Saponin Extract from Soapnuts” by teaching following process: First, crush the soapberry fruit into large pieces and soak them in water. Second, treat the soapberry liquid with an aerated floating process to produce bubbles. Third, collect the bubbles and let them set to form saponin extract. In this case, the saponin extract will be undamaged. Also, this method is an upgrade over the traditional process of stewing soapberry in which the saponin extract was often damaged.

### SUMMARY OF THE INVENTION

Methods as disclosed in both cited documents have following drawbacks:
1. As disclosed in cited reference No. 1, organic acid is removed by an ions-exchange process. However, such abundant organic acid is critical for producing saponin extract. Furthermore, the sugar content in the fruit itself is used to produce saponin, and therefore using weak ion-exchange process to remove its sugar content will also reduce the benefits of soapberry.
2. In cite reference No. 2, it discloses “the soapberry liquid is treated with aerated floating process to produce bubbles, and the bubbles are collected and placed in quiescent state to form saponin extract.” The collection process requires large space for the storing of air bubbles, thus, making the process very uneconomical. Furthermore, soapberry saponin is a kind of active saponin, and unlike soap made by petrochemical raw materials, soapberry saponin once used, is unable to re-foam and thus is useless as a commercial product. Therefore, to extract soapberry saponin more effectively for commercial use has become the first objective of the present invention.
3. Further, the efficiency of above methods are low and unable to extract the soapberry active interface saponin, organic acid, organic vitamins, and glucose enzyme, as well as oleic alcohol from soapberry seeds completely. Also, it is impossible to bring soapberry saponin useful functional effects into practice. In view of this, how to effectively improve the efficiency of production and completely bring out its useful organic ingredients, has become another objective of the present invention.
4. Moreover, the method as disclosed in cited reference No. 2 is unable to extract all the useful organic ingredients of soapberry; only a single kind of product can be produce by this method, thus have low potential of product diversification. Therefore, in view of its limited usefulness and economic efficiency, how to effectively extract all the useful organic ingredients from soapberry, as well as to achieve product diversification is the third objective of the present invention.
   The present invention relates to a method that is free from any liquid or solid waste, carbon emission and chemical pollution, and also with low-power consumption for extracting active interface saponin, organic acid, organic vitamins, and glucose enzyme as well as oleic alcohol products from the process of fermentation and create a finished product. Said method includes following steps:

Step 1: Initial fermentation process. Separate raw Soapberry fruits (1) into pericarps (11) and seeds (14), and place said pericarps (11) in a container, then stay under a temperature in range of 20° C. to 90° C. for half to thirty-six months to make them fermented naturally.

Step 2: Initial cleaning process. The pericarps (11) fermented in step one are placed inside a sealed tank (2), fill water into said sealed tank (2), then stand for 5-10 minutes to make the foreign object attached on said pericarps (11) being dissolved or moved by the water, supersonic vibrator or shifting the pressure inside said sealed tank (2) could be applied for speeding up the detachment of the foreign object on said pericarps (11), finally, take the pericarps (11) out of the tank (2), and the liquid left in the tank (2) become product 1 (10) which contains enzyme produced in step 1 by fermenting.

Step 3: Put cleaned soapberry pericarps (11) into a blender (3) to grind them into the shattered mix of the pulp (12) and fruit fiber (13).

Step 4: Soak said shattered mix of the pulp (12) and fruit fiber (13) with purified water, and pour them into an extractor (4). Activate the extractor, and said pulp (12) will be drained out of the extractor with said purified water, the water contains said pulp (12) produced by this step is called soapberry liquid (A) hereafter; and the fruit fiber (13) left in the extractor become product 2 (20).

Step 5: Said soapberry liquid (A) extracted from step four is pumped into a stewing pot (5) and heated for eliminating comprised bacteria by high temperature.

Step 6: Carry out second fermentation process. Said soapberry liquid (A) decontaminated in step five is pumped into a vacuum device (6) and placed for half to six months to execute a second fermentation, thereby forming soapberry syrup (D) containing interface active saponin, organic acids, glucose and organic vitamins therein.

Step 7: Pumping the soapberry syrup (D) from a vacuum device (6) to a container (7) with constant air pressure; stand for half to six months, until the flesh (C) all sunk to the bottom of the container (7), and the liquid part of said soapberry syrup (D) become the pectin (B), thus, said flesh (C) and pectin (B) become product 3 (30) and product 4 (40) correspondingly. A temporary storage device (71) is disposed between the vacuum device (6) and the container (7) for balancing the pressure difference gradually.

Step 8: Pumping the soapberry pectin (B) from the container (7) into a sedimentation tank (8) for allowing the pectin (B) being further separated into a purified pectin (E) and soapberry paste (F) which is formed by sedimented solid part of soapberry, and correspondingly become product 5 (50) and product 6 (60).

Furthermore, step 9 which further includes steps 9-1 to 9-3, is introduced as following:
9-1: The purified pectin (E) and the soapberry paste (F) from the sedimentation tank (8) are guided into a drying device (9A) separately for dehydrating excess water into aponin crystalline powder (G) and saponin powder (H) which are formed into product 7 (70) and product 8 (80) respectively.
9-2: The purified pectin (E) is guided from said sedimentation tank (8) into a distillation device (9B) for producing sapogenol (I) by distillation, which is product 9 (90).
9-3: Said product 6 (60) (soapberry paste (F)) is guided into a pyrolysis and activating device (9C) for being carbonized and activated to be produced as product 10 (500) which is an active carbon saponin powder (J).

The product 1 (10) produced by step 2 can be used for irrigating crops, phytopathology treatment or soil reviving; the product 2 (20) produced by step 4 is a fertilizer for plants, a cultivating soil or a recycled raw material; the product 3 (30) and product 4 (40) produced by step 7, are with low purity of saponin, which could be the raw material of shower gel, tableware cleaner, soap and skin care products; the product 5 (50) and product 6 (60) produced by step 8, are with higher purity of saponin, which could be the raw material of high value cleaning and skin caring products, and medicine; The finished products 7 (70) and product 8 (80) produced by step 9-1 are with highest purity, which could be the raw material of luxury cleaning and skin caring products, and medicine. The product 9 (90) produced by step 9-2 could be the raw material of post-distillation soapberry alcohol, which could be used for medical purpose and the non-erosive solution that is applied for skin care products. The product 10 (500) produced in step 9-3 could be the raw material activate-carbonate saponin powder.

The soapberry seeds (14) separated in step 1 of claim 1 can also be processed by following steps:

Step 1: said soapberry seeds (14) extracted from the soapberry fruits (1) are placed into a heating device (9D) and heated for half to six hours within 45° C. to 98° C. for activating an oleic alcohol therein.

Step 2: said activated soapberry seeds (14) through step one are placed into a shell-breaking device (9E) for a coarse-crushing process to separate shelves (15) and nuts (16).

Step 3: the soapberry nuts (16) are placed into a compressor (9F) for extracting crude oil (100) and coarse nut residual (200).

Step 4: the crude oil (100) is guided into a centrifugal separation device (9G) for separating oleic alcohol essences (300) with different densities.

Further, a step 5 can be included: coarse nut residuals (200) are placed into a milling device (9H) for grinding the residual (200) into soapberry powder (400).

Further, a step 6 can be included: said soapberry powder (400) is placed into a molding device (91) for generating a product 11 (600). Said product 11 (600) is skin-care mask.

### COMPARISON OF THE PRIOR ART

The following advantages can be obtained in accordance with the present invention.
1. Soapberry is very rich in natural chlorophyllin fructose amino acids. Therefore, soapberry is definitely acidic, but not alkaline. Because soapberry contains traces of poisonous acid, therefore has to go through several stages of bio-tech treatment before it can be used. For these reasons, present invention therefore applies continuous fermentation (brewing fermentation). The natural yeast particles produces during the fermentation process can decompose high-sugar, and the “steady-state” fructose amino acid can then be released. Finally, soapberry saponin is obtained. The entire process is very similar to the process of making wine. No preservatives (such as ethylene acid) or microbial antibacterial agents are required to prevent unwanted bacteria from decomposing or rotting. Compared to either traditional methods or the method described in cited document No. 2, the present invention offers many more practical benefits.
2. Moreover, no chemical additive is added during the entire manufacturing process, as well as its follow-up treatment. With this, the fully ripen soapberry pulps will then go through a process whereby pectin, flesh and fiber are separated and extracted. The high glucose, polysaccharide soapberry is used as a raw material that undergoes an alcoholization-fermentation-saponin process. After this, different production technologies of differing grades are employed to further separate the raw material into pectin, flesh, paste, fiber and so on, all of which contain saponin. The purpose of present invention is to make sure that the finished products are healthy, toxin free, and biologically safe, and that they produce no liquid or solid waste, zero pollution, low energy consumption, zero carbon emission, zero pollution, and are environmental-friendly.
3. Said soapberry syrup (D) extracted by the present invention, can restrain the activity of the bacteria or tyrosinase, providing medical or cosmetic features. By adding the function of saponin, the soapberry syrup (D) is more effective than most synthetic products, suitable as a replacement.
4. Extract soapberry seeds (14) from the fruit (1) and produce raw materials for crude oil, coarse nut residual, alcohol essential oil products, fine soapberry powder and so on base on the processing method of the present invention. This complete utilization of the left over soapberry seeds (14) from the processing of pulps (11) and flesh (12) of soapberry (1) means the maximum application of the entire fruit (1). By doing so, we can attain the goals of zero solid and liquid waste, zero carbon emission, and low-power consumption.
5. All forms of waste produced during the manufacturing process as disclosed in the present invention are re-usable. Finished products are diversified and cover a wide range: from daily necessities, agricultural supplies, medical supplies, construction supplies to food ingredients, and so on; thus reducing overall costs to achieve maximum economic benefit.

### BRIEF DESCRIPTION OF DRAWINGS

FIG. 1 is a flow diagram illustrating the method for extracting substances from soapberry fruits.

FIG. 2 is a flow diagram illustrating the reprocess of the method of FIG. 1.

FIG. 3 is a flow diagram illustrating the method for extracting substances from soapberry seeds.

FIG. 4 is a flow diagram illustrating the reprocess of the method of FIG. 3.

### DETAIL DESCRIPTION OF PREFERRED EMBODIMENTS

The description of preferred embodiment is described in detail according to appended drawings hereinafter.

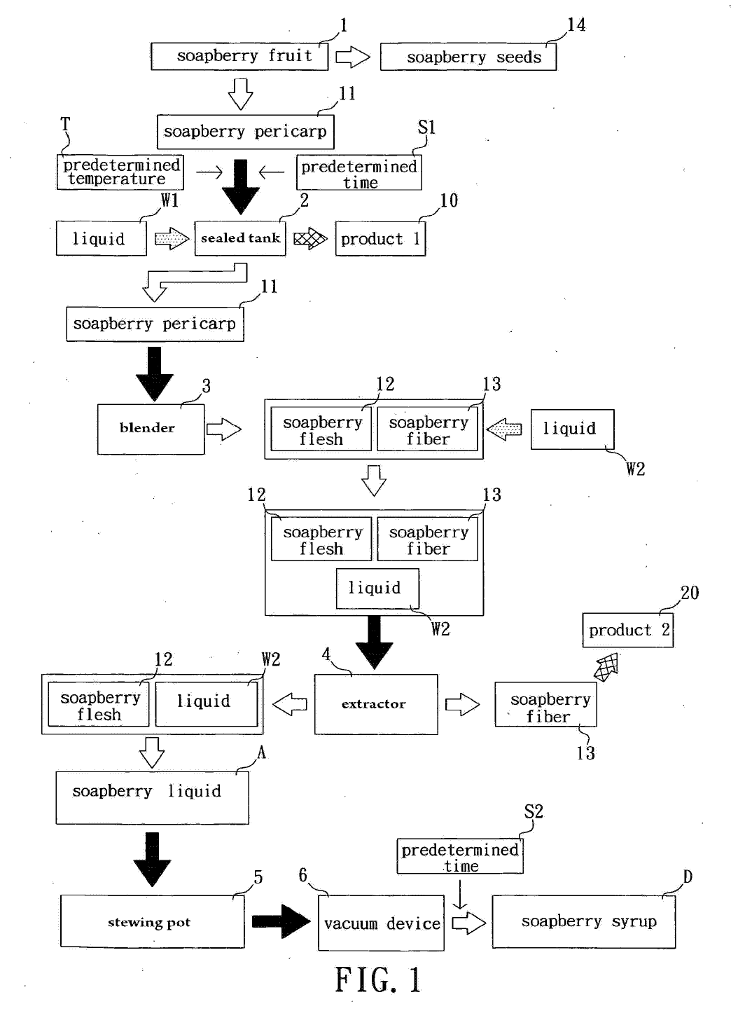

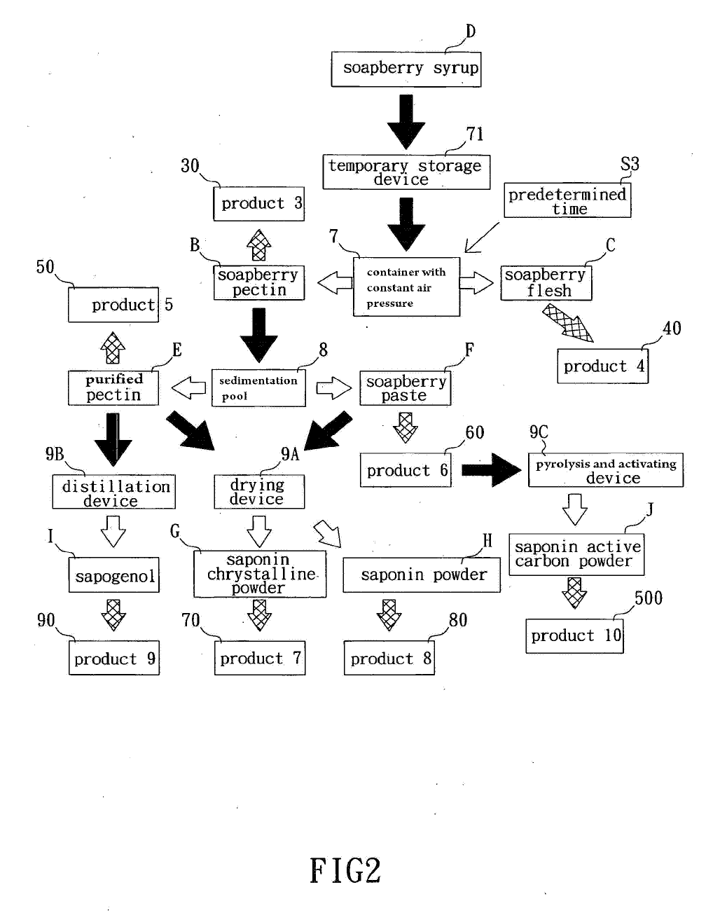

Referring to FIGS. 1 and 2, a method for extracting substances from soapberry fruits is characterized in that including following steps:

Step 1: separate raw Soapberry fruits into pericarps and seeds, and store said pericarps under a constant temperature (T) for a predetermined time (S1) to make them fermented naturally;

Step 2: put the pericarps fermented in step one inside a sealed tank, than fill in said sealed tank with liquid (W1), then stand for 5-10 minutes, to make the foreign object attached on said pericarps being dissolved or moved by said liquid (W1), supersonic vibrator or shifting the air pressure inside said sealed tank could be applied for speeding up the detachment of the foreign object on said pericarps, finally, take the pericarps out of the tank, and the liquid left in the tank becomes product 1 which contains enzyme produced in step 1 by fermenting;

Step 3: Put cleaned soapberry pericarps into a blender to grind them into the shattered mix of the pulp and fruit fiber;

Step 4: Soak said shattered mix of the pulp and fruit fiber with liquid (W2), then pour them into an extractor; activate the extractor, and said pulp will be drained out of the extractor with said liquid (W2), the water contains said pulp produced by this step is called soapberry liquid hereafter; and the fruit fiber left in the extractor become product 2;

Step 5: Said soapberry liquid extracted from step four is pumped into a stewing pot and heated for eliminating comprised bacteria by high temperature;

Step 6: said soapberry liquid decontaminated in step five is pumped into a vacuum device and placed for a predetermined time S2 to process a second fermentation, thereby transforming into a soapberry syrup containing interface active saponin, organic acids, glucose and organic vitamins therein;

In order to achieve zero solid and liquid waste, zero carbon emission, zero pollution, low-power consumption, as well as to protect environment, said method is processed under a “Bioscience Tristate” (pure-state, stable-state, and ripen-state) production technology to maximize use of the entire soapberry. In other words, an Alcoholization-Fermentation-Saponin biotechnological manufacturing technique is used to produce soapberry (1) products.

The above-mentioned “Pure-state” refers to a pure and organic soapberry fruit (1) undergoing pectin, flesh and fiber processing as well as extraction, which is clearly illustrated in step two and three.

Furthermore, the “ripened-state” refers to the management of finished products so that the soapberry (1) can undergo (biodegrading, fermenting, and catalysis) process under natural environment and normal climatic temperature. With this, the original yeast strain can give rise to a single type of biological species of microzyme, and the microzyme can then produce adeno-sine triphosphate, and the active effect of adenosine diphosphate can cause the original color, flavor, taste, and also the special polysaccharides composition of that particular material becomes richer. Also, by releasing the organic energy, it can remit the need to use preservatives. In other words, this refers to the fermentation process mentioned in step 1 and 6.

Through the “Bioscience Tristate” processing, the useful components of soapberry can be decomposed and made into the following types of products: saponin fruit glue, saponin pulp, saponin paste, saponin fiber and others substances. All these raw materials are able to achieve the goals of promoting healthy, non-toxic, and safe products.

Going through the manufacturing process of this Innovation, the amino acid is released from the soapberry fruit (1), and the safe, non-toxic, active and vitalizing proteins can then be extracted therefrom. The nutrition of multi-vitamins can also be used as natural raw materials, which all these can prevent the harm from chemical toxin.

Said product 1 (10) and 2 (20) generated in the disclosed method are treated as liquid and solid waste traditionally as well as by the method adopted by cited document 2 mentioned above. However, in the present invention where the biological Tristate manufacturing technology is used, product 1 (10) and product 2 (20) are not waste without value. Said product 1 (10) can be used for irrigating crops, phytopathology treatment or soil reviving; and product 2 (20) is a fertilizer for plants, a cultivating soil or a recycled raw material.

Product 1 can be used as an insect repellent or environmental regeneration medication; while product 2 can be used as agricultural fertilizer or soil cultivator. Both these products can replace petrochemical pesticides, and with their rehabilitative capability, these products can help resolve potential pollution problems in the growing of crops. For example, [1] metallic and non-metallic pollution, [2] nitrate and nitrite pollution, and [3] pesticide pollution can all be completely avoided so that fruits and vegetables can grow into genuine green food. Also, by using product 2 (20), the soil cultivator, improved dwarfing can be done to fruit trees and veggies, and vegetation can be harvested at a shorter height.

Generally speaking, agricultural planting covers the following two methods of cultivation and fertilization:
1. Organic-planting: Using bare land or soil tillage as the plating foundation and mixing them with decay organic substances that possess organic and biological function for fruits and vegetables planting. The fertilizer of this planting include collecting all kinds of kitchen waste such as fishes, meats, fruits and veggies and make them into compost. By means of “micro-structures technology”, these wastes will then undergo a decomposing process to produce liquid and solid fertilizers for vegetation purposes. The main objective of this method is to protect the environment by using renewable resources.
2. “Pure-state planting”: Using plants fibers as the planting ground and make use of the unique “implant-structure” of plants as well as the “pure properties” of plants extracts for planting. The fertilizer of this kind include the use of the unique property (herewith referred to as the “pure quality”, for example, the fructose amino acid and active interface of soapberry) of a single plant species to enhance the growth of crop strains, prevent “organism infection”, enhance protection from bacteria, pests, and “eco-organism”. The crop strains, having absorbed these unique substances, will provide more nutrition and better curative effects to the grown-up fruits and veggies, and thus, make better food for mankind.

Said product 2 (20) can serve as the fertilizer of “pure-state planting”. We can achieve “pure-state planting” requirements because both the fructose acid and the special active interface of soapberry are what we termed “pure quality” as mentioned above.

Moreover, the product 2 (20) can also be used as environmental-friendly renewable materials. For example, three-plywood and laminating board. This not only can help reducing the overall manufacturing cost, but also can help avoid deforestation. Moreover, because product 2 (20) contains small amount of saponin, it can thus reduce significantly the ration of pest breed, and can made into very good quality environmental renewable materials.

Said method further includes a step 7, which is as following:

Pumping the soapberry syrup (D) from a vacuum device (6) to a container (7) with constant air pressure; stand for half to six months, until the flesh (C) all sunk to the bottom of the container (7), and the liquid part of said soapberry syrup (D) become the pectin (B), thus, said flesh (C) and pectin (B) become product 3 (30) and product 4 (40) correspondingly. A temporary storage device (71) is disposed between the vacuum device (6) and the container (7) for balancing the pressure difference gradually.

Further, a step 8 is also included, which is as following:

Pumping the soapberry pectin (B) from the container (7) into a sedimentation tank (8) for allowing the pectin (B) being further separated into a purified pectin (E) and soapberry paste (F) which is formed by sedimented solid part of soapberry, and correspondingly become a product 5 (50) and a product 6 (60).

Further, a step 9-1 is included, wherein the fine pectin (E) and the soapberry paste (F) from the chromatography filtration device (8) are guided into a drying device (9A) separately for dehydrating excess water, into a saponin crystalline powder (G) and a saponin powder (H) which are formed into a product 7 (70) and a product 8 (80) respectively.

Further, a step 9-2 is included: the fine pectin (E) is guided from said chromatography filtration device (8) into a distillation device (9B) for distilling a sapogenol (I) and which is product 9 (90).

Further, a step 9-3 is also included: said product 6 (60) is guided into a pyrolysis and activating device (9C) for being carbonized and activated to be produced as a product 10 (500) which is an active carbon saponin powder (J).

Said steps 7 to 9-2 are known as “stable-state”. This stable-state Alcoholization-Fermentation-Anhydride (AHA) biotech manufacturing method made use of the high glucose polysaccharide physical nature of soapberry to undergo protein settlement dialysis, distillation and filtration, detoxification by using high temperature positive-negative pressure and speed up alcoholization in fermentation. Once the multi-carbohydrate is released, an intermittent period is required for a qualitative change in the process of re-fermentation. At this stage, proliferative expansion by the original yeast molecules will occur. Once reaching a certain pressure, the original yeast molecules will start to contract, forming a vacuum within the device, and saponin liquid of different grades is produced and made into different products.

The end products manufactured by step 7 are product 3 (30) and product 4 (40), wherein the product 3 (30) can be made into unrefined pectin and product 4 (40) can be made into unrefined soapberry flesh. Both products (30,40) have average purity, which can be used as ordinary soapberry saponin cleanser and skin care products, or saponin soapberry soap.

The end products manufactured by step 8 are product 5 (50) and product 6 (60), wherein said product 5 (50) can be made into purified pectin, and product 6 (60) can be made into soapberry paste. Both products (50,60) have higher purity, which can be used as higher quality soapberry saponin cleanser and skin care products, or saponin soapberry soap. This can even be made into general medical drugs.

The end products manufactured by step 9-1 are product 7 (70) and product 8 (80), wherein said product 7 (70) can be made into saponin crystalline powder, and product 8 (80) can be made into saponin power. Said products (70, 80) have the highest purity, which can be used as the best quality soapberry saponin cleanser and skin care products, or saponin soapberry soap. This can even be made into higher quality medical drugs that yield best effect.

The end product manufactured by step 9-2 is product 9 (90) which can be made into raw materials for post-distillation soapberry alcohol. Said product (90) contains sapogenol (I) which is a kind of natural botanical interface alcohol solution which can be used for medical purposes or non-corrosive solvent for skin care products. It can also replace commonly used alcohol, reduce or avoid harm to human body, or even side effects.

As aforementioned, from product 3 (30) to product 9 (90), due to the difference in extracting method and quality, they can therefore be made into different grades and types of cleansing and skin care products.

Cleansing products are: fine optical component cleansing fluid, fine electronic cleansing component fluid, heavy metals cleansing fluid, ordinary daily cleansing fluid, ordinary fruits and veggies cleansing fluid and so on.

Skin care products are: shower gel, facial gel, shampoo, bath powder, soap, facial mask, night-cream and so on. Skin care products in this Innovation cover an extremely wide spectrum and can replace all types of skin care products. Products in present invention are natural, pollution free, and with better efficacy.

There are some differences in the properties of cleansing products. In present invention, they are categorized into the following 2 items:
1. Inorganic detergent: Suitable for cleansing tableware, clothing and industrial equipment. Such cleansing agent can breakdown and decompose either acidic or alkaline chemical compound of inorganic objects, oxidize the rusts on appliances or utensils. However, such detergent cannot be directly used for cleaning up of organic substances.
2. Organic detergent: Suitable for cleansing organic substances. Organic detergent can be used for biodegrading pesticide residues in fruits and veggies, and remove the toxicity. Furthermore, it can also use as human or even pets' body cleanser, skin care and maintenance products. The organic elements of soapberry can effective clean and care for the human and pets' skin simultaneously, but consumers must know how to identify genuine and fake soapberry products to ensure the best security.

Soapberry extract contains abundance fructose amino acids and organic active interface. Besides safety, environmental friendliness and non-toxicity, the abundant nutrients of its extracts can also provide sufficient nourishments for the tissue cells of organisms, thus active the cells and give them enough energy to exercise the course of catalyze metabolism so as to achieve the purposes of cleanliness and health care.

As for the use of medical drugs, according to the classic Chinese medicine book, such as Compendium of Materia Medica, and modern experimental reports and clinical trials, soapberry has long been proven to have medical efficacy which can be used to treat cardiovascular and cerebrovascular diseases. It is also anti-bacterial, anti-inflammatory, antitussive, antiasthmatic, and anti-phlegm.

Furthermore, based on the raw materials with different saponin densities, they can be made into granules, capsules, tablets, suppositories and other medicine type. They can be used as formulations for clinical treatment of hypertension, angina, high blood lipids, stroke sequelae, chronic gastritis, inflammatory disease such as antimicrobial, and so on. On the other hand, it can also serve as raw materials for antibiotics. Recently, it is even discovered that soapberry has a certain degree of efficacy for treating cancer. Therefore, its use is very extensive in the medical profession. And the most importantly, the curing content in said drugs is purely natural but not synthetic.

In addition, product 5 (50), product 6 (60), product 7 (70) and product 8 (80) can even be used as the raw of fire foam fluid which can replace petrochemical-based fire foam fluid, and can also be used as extension agent. Due to their saponin from soapberry, the fire foam fluid made of the said products can therefore attain a better effectiveness than what is made of the petrochemical raw materials, and can also prevent the fire from spreading under elevated temperature. Furthermore to all these, it will not cause chemical pollution to the environment.

The products generated by the method as disclosed can be diversified in different grades of products depending on different purities of extracts, and the well-structured nature is very similar to that of the petrochemical industry. However, the most prominent differences between the two is that in this Innovation, a totally pollution free can be attained in both the environment as well as the manufacturing process. The products thus provide 100% environmental protection and cause no harm.

Furthermore, product 3 (30) and product 5 (50) are similar to that of product 1 (10) can be served as liquid for soil reviving or environmental regeneration, providing different levels of effectiveness. The reason is that the extracts from the method contain organic substances, and the only difference lies in the different levels of the purity of said extracts.

Therefore, product 1 (10), product 3 (30) or product 5 (50) can all be used as regeneration water for vegetation. Pertaining to lands chemically polluted by heavy metals like chromium, lead, cadmium, arsenic, mercury and other types of harmful substances, so long as said pure or water-diluted products of this Innovation be infiltrated about one to three meters below the ground, the organic substances of the soapberry extracts can eliminate the toxic substances and heavy metals to achieve the goal of bioremediation. Also, the products of present invention are more effective than the conventional phytoremediation, due to the effect of the products of present invention are not affected by the climate, irrigation, fertilization and other factors. Moreover, the effect occurs sooner and the price is cheaper, thus, by the application of said products of present invention, the soil remediation of pollutants as well as the maintenance of soil properties can both be taken care of.

Furthermore, as compared to the so-called soil conditioner used nowadays, products from the present invention are genuinely natural with zero chemical composition, and thus will not lead to groundwater contamination. If said products are used together with soil conditioner, not only can it enhanced rhizosphere effect, but can also speed up the rate of land restoration significantly and shorten the time for land restoration. Also, by recovering the environment in a natural manner, we can then stop further environmental impact from mankind.

According to the afore-mentioned, a temporary storage device (71) must be placed in between the vacuum device (6) and container (7) for storing soapberry syrup (D) temporarily until next process initiating and balancing the pressure difference between the vacuum device (6) and the container (7); Temporary storage of the syrup (D) to empty the vacuum device (6) for a subsequent vacuum operation, ensuring that the entire manufacturing process can operate without any interruption.

Said predetermined time (S1) is in a range of half to thirty-six months. Both predetermined time (S2) and (S3) are in a range of half to six months. The temperature (T) for said predetermined time (S1, S2, S3) are in a range of 20° C. to 90° C. Adequate time and constant room temperature must be provided so that the fruit (1) as well as its end products can undergo fermentation process and release their organic energy as well as for obtaining the best efficacy and for easy extraction.

Said liquid (W1) as mentioned in step 2 is water, and liquid (W2) as mentioned in step 4 is purified water. This patent uses only water for the entire manufacturing process. Said liquid (W1) used for cleaning contains enzyme amino acid during the initial fermentation process, therefore, it can be used as organic agent for preventing phytopathogen. The method disclosed in the embodiment produces no waste and is zero pollution, thus, makes it an ECO-Friendly manufacturing method.

A step 9-3 is further included in the manufacturing method, wherein said product 6 (60) is guided into an activated-carbon production device (9C) for being carbonized and then becomes a product 10 (500) which is a saponin active carbon powder (J).

Said product 10 (500) doesn't have only outstanding absorbing effects, but also keeps the whole features and benefits of saponin, which makes it contributing more effect than conventional active carbon powder.

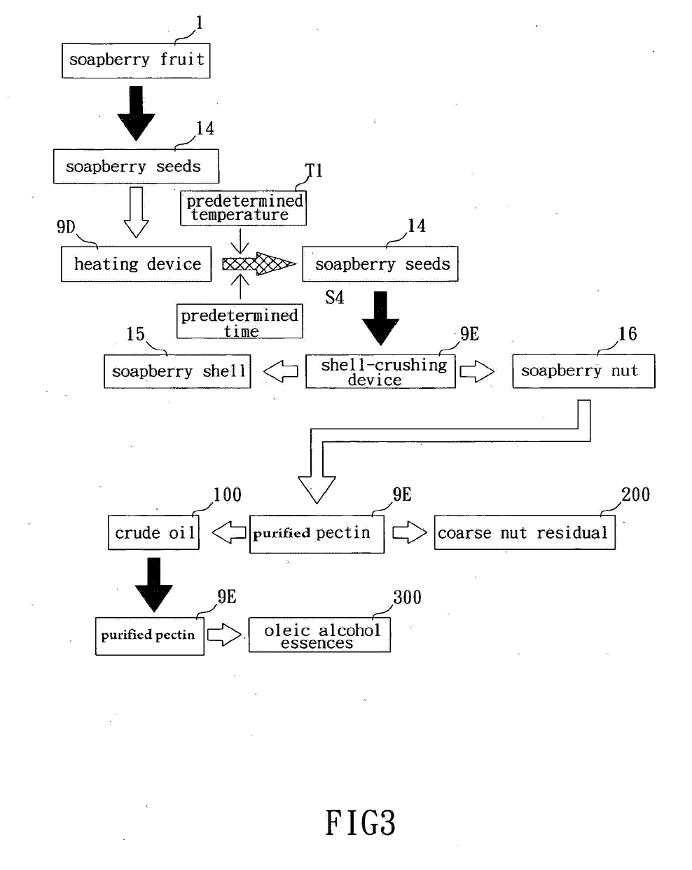

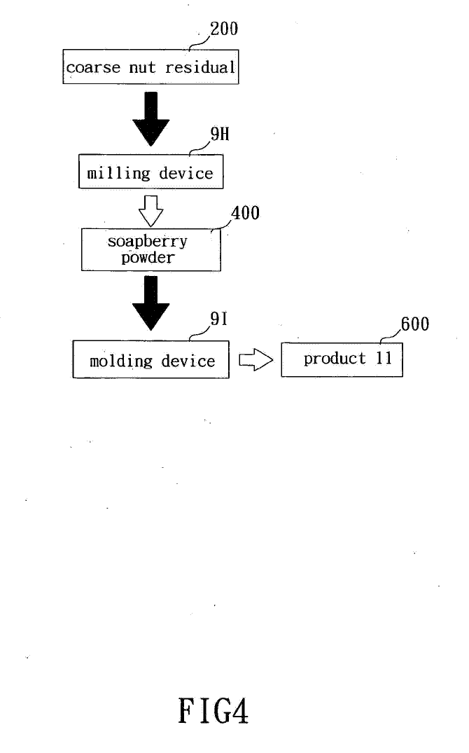

As mentioned above, said method produce no waste and by referring in FIGS. 3 and 4, a method for extracting substances from soapberry seeds are illustrated which includes following steps:

Step 1: said soapberry seeds (14) extracted from the soapberry fruits (1) are placed into a heating device (9D) and be heated for a predetermined temperature (T1) within a predetermined time (S4) for activating an oleic alcohol therein.

Step 2: said activated soapberry seeds (14) through step 1 are placed into a shell-breaking device (9E) for a coarse-crushing process to separate shelves (15) and nuts (16).

Step 3: the soapberry nuts (16) are placed into a compressor (9F) for extracting crude oil (100) and coarse nut residual (200).

Step 4: the crude oil (100) is guided into a centrifugal separation device (9G) for separating oleic alcohol essences (300) in different densities.

Said soapberry shelves (15) produced during step 2 not only can be treated as raw materials for the subsequent process, but can also be used as fertilizers for crops, cultivation soil, or recycled raw materials. The soapberry nut (16), other than used as raw materials for the subsequent process, can also be treated as raw materials of feed for cultivation.

Moreover, oleic alcohol essences (300) produced by the method, not only can be treated as raw materials for a subsequent process, but can also be used as aesthetics medicine (antioxidant for epidermal tissue and moisturizing cortical cells) as well as being used as medical supplements (glycerol alternative for basic oil), or even cooking oil for health purposes.

Said predetermined time (S4) is in a range of thirty minutes to six hours; and the predetermined temperature (T1) is in a range of 45° C. to 98° C. By providing sufficient time and appropriate temperature, the oleic alcohol extracted from soapberry seeds (14) become activated, and the seeds (14) release all their organic elements, oleic alcohol during the subsequent processing.

Said method for extracting substances from soapberry seeds further includes a step 5, wherein coarse nut residuals (200) are placed into a milling device (9H) for grinding the residual (200) into soapberry powder (400). The soapberry powder (400) produced during said step 5 not only can be treated as raw materials for the subsequent process, but also can be used as additional ingredients for bakery and cookies. Furthermore, it can also achieve the goal of lesser oil consumption as well as served as nutritional additives for various kinds of organic, high fiber health care products.

Said method for extracting substances from soapberry seeds further includes a step 6, wherein said soapberry powder (400) is placed into a molding device (91) for generating a product 11 (600), which is skin-care mask.

They can be used as health care products such as facial mask. Moreover, having directly molded as final product, neither tissue paper nor petrochemical plastic molding is required, therefore ensuring a healthier and safer environment.

What is described and presented with figures is only the main principle and better embodiments of present invention, what is verified and modified by the scope of present invention, may meet what is claimed hereafter.

What is claimed is:

1. A method for extracting substances from soapberry fruit and its seeds, comprises following steps:
Step 1: separate raw Soapberry fruits into pericarps and seeds, and store said pericarps under a constant temperature (T) for a predetermined time (S1) to make them fermented naturally;
Step 2: put the fermented pericarps in step one inside a sealed tank, than fill in said sealed tank with liquid (W1), then stand for 5-10 minutes, to make the foreign object attached on said pericarps being dissolved or moved by said liquid (W1), supersonic vibrator or shifting the air pressure inside said sealed tank could be applied for speeding up the detachment of the foreign object on said pericarps, finally, take the pericarps out of the tank, and the liquid left in the tank becomes product 1 which contains enzyme produced in step 1 by fermenting;
Step 3: Put cleaned soapberry pericarps into a blender to grind them into the shattered mix of the pulp and fruit fiber;
Step 4: Soak said shattered mix of the pulp and fruit fiber with liquid (W2), then pouring them into an extractor; activate the extractor, and said pulp will be drained out of the extractor with said liquid (W2), the water contains said pulp produced by this step is called soapberry liquid hereafter; and the fruit fiber left in the extractor become product 2;
Step 5: Said soapberry liquid extracted from step four is pumped into a stewing pot and heated for eliminating comprised bacteria by high temperature;
Step 6: said soapberry liquid decontaminated in step 5 is pumped into a vacuum device and placed for a predetermined time S2 to process a second fermentation, thereby transforming into a soapberry syrup containing interface active saponin, organic acids, glucose and organic vitamins therein;

2. A method for extracting substances from soapberry fruit and its seeds defined in claim 1, wherein said predetermined time (S1) is in a range of half to thirty-six months and predetermined time (S2) is in a range of half to six months; said liquid (W1) is water, and liquid (W2) is purified water; and the temperature (T) is in a range of 20 to 90° C.

3. A method for extracting substances from soapberry fruit and its seeds defined in claim 1, wherein said soapberry syrup is processed by step 7, which is as following:
Pumping the soapberry syrup from a vacuum device to a container with constant air pressure; stand for half to six months, until the flesh all sunk to the bottom of the container, and the liquid part of said soapberry syrup become the pectin, thus, said flesh and pectin become product 3 and 4 correspondingly; a temporary storage device is disposed between the vacuum device and the container for balancing the pressure difference gradually.

4. A method for extracting substances from soapberry fruit and its seeds defined in claim 1, wherein said pectin is processed by step 8, which is as following:
Pumping the pectin from the container into a sedimentation tank for allowing the pectin being further separated into a purified pectin and soapberry paste which is formed by sedimented solid part of the soapberry, and correspondingly become a product 5 and 6.

5. A method for extracting substances from soapberry fruit and its seeds defined in claim 3, wherein said product 5 and 6 are processed by step 9-1, which is as following:
The product 5 and 6 from the sedimentation tank are guided into a drying device separately for dehydrating excess water, into a saponin crystalline powder and a saponin powder which are product 7 and 8 respectively;

6. A method for extracting substances from soapberry fruit and its seeds defined in claim 3, wherein said product 5 is processed by step 9-2, which is as following:
The product 5 is guided from said sedimentation tank into a distillation device for producing sapogenol by distillation, which is product 9.

7. A method for extracting substances from soapberry fruit and its seeds defined in claim 3, wherein product 6 is processed by step 9-3, which is as following:
The product 6 produced by said step eight, is guided into an pyrolysis and activating device for carbonizing and activating said product 6 to generate a product 10 which is an active carbon saponin powder.

8. A method for extracting substances from soapberry fruit and its seeds defined in claim 1, wherein the seeds separated in stepl, are processed by following steps:
Step 1: place soapberry seeds extracted from the soapberry fruit into a heating device and be heated for a predetermined temperature (T1) within a predetermined time (S4) for activating the oleic alcohol therein;
Step 2: place heated soapberry seeds into a shell-breaking device for a coarse-crushing process to separate shelves and nuts;
Step 3: place said soapberry nuts into a compressor for extracting the crude oil and coarse nut residual;
Step 4: guide the crude oil into a centrifugal separation device for separating oleic alcohol essences in different densities.

9. A method for extracting substances from soapberry fruit and its seeds defined in claim 8, wherein said predetermined time (S4) is in a range of half to six hours; and said predetermined temperature (T1) is in a range of 45 to 98° C.

10. A method for extracting substances from soapberry fruits and seeds defined in claim 8, wherein said nut residual is processed by step 5, which is as following:
Place coarse nut residuals into a milling device for grinding the residual into a soapberry powder.

11. A method for extracting substances from soapberry fruit and its seeds defined in claim 10, wherein said soapberry powder is processed by step 6, which is as following:
Place said soapberry powder into a molding device to form a skin-care mask which is product 11.
